(19) United States
(12) Patent Application Publication
(10) Pub. No.: US 2004/0202718 A1
(43) Pub. Date: Oct. 14, 2004

(54) Dosage form for treatment of diabetes mellitus

(76) Inventors: Ziauddin Z. Tyebji, Baroda (IN); Harivardhan L Reddy, Baroda (IN)

Correspondence Address: TIMOTHY J MARTIN, PC, 9250 W 5TH AVENUE, SUITE 200, LAKEWOOD, CO, 80226, US

(21) Appl. No.: 10/491,305

(22) PCT Filed: Sep. 27, 2002
(86) PCT No.: PCT/IN02/00194

(30) Foreign Application Priority Data
Sep. 28, 2001 (IN) 941/MUM/2001
Sep. 28, 2001 (IN) 942/MUM/2001

(51) Int. Cl. A61K 9/24

(52) U.S. Cl. 424/471

(57) ABSTRACT

A dosage form for the treatment of diabetes mellitus and conditions associated with it comprising a biguanide such as metformin or its pharmaceutically acceptable salt wherein the metformin is released in a controlled manner. A dosage form for the treatment of diabetes mellitus and conditions associated with it comprising an immediate release composition comprising a long-acting sulfonyl urea and a controlled release composition comprising a biguanide.

The present invention relates to a dosage form for treatment of diabetes mellitus and conditions associated with it comprising a biguanide such as metformin or its pharmaceutically acceptable salt wherein the metformin is released in a controlled manner.

The present invention also relates to a dosage form for treatment of diabetes mellitus and conditions associated with it comprising a combination of long-acting sulfonyl urea and a biguanide such that the long-acting sulfonyl urea is released immediately and the biguanide is released in a controlled manner.

### BACKGROUND OF THE INVENTION

a) Introduction:

Non-insulin dependent diabetes mellitus (NIDDM), also known as maturity-onset diabetes or diabetes mellitus type 2, is a frequent metabolic disease and the main cause of hyperglycemia. It is a heterogeneous disease with complex, unclarified metabolic aspects. Insulin secretion may appear normal or even excessive, but it is insufficient to compensate for insulin resistance. The disease is characterized by three main abnormalities of metabolism contributing to hyperglycemia. These include the partial or complete decrease in insulin secretion, resistance of the peripheral tissues to insulin and increased hepatic production of glucose in fasting conditions. Diet and physical exercise cause a reduction in insulin-resistance and lead to an improvement in the pancreas deficit over a period of time.

When these provisions are not sufficient, a pharmacological agent(s) needs to be taken for control of hyperglycemia. Sulfonyl ureas and biguanide derivatives have been used in diabetes therapy. The use of these classes of compounds in the monotherapy has been effective in obtaining a glycometabolic control in diabetic patients.

Biguanide derivatives like metformin, phenformin and buformin, generally in the form of their hydrochloride salt, have been used as anti-hyperglycemic agents in the treatment of non-insulin dependent diabetes mellitus. The mechanism of action of the drugs belonging to this class includes reduction in hepatic glucose production, decrease in intestinal absorption of glucose, and increase in glucose uptake and utilization. Biguanides improve glucose tolerance in patients with diabetes mellitus type 2, lowering both basal and post-prandial plasma glucose. With biguanide therapy, insulin secretion remains unchanged while fasting insulin levels and day-long plasma insulin response may actually decrease. Although phenformin is still used widely as an anti-hyperglycemic agent, metformin is the preferred biguanide, as it exerts a better normoglycemic action with a lower risk of lactic acidosis—a common side-effect with phenformin therapy. Metformin is also known to lower blood triglyceride levels and assist in weight reduction.

b) Controlled Release Metformin Compositions:

Metformin hydrochloride is highly soluble in water aid has intrinsically poor permeability in the lower portion of the gastrointestinal tract and an elimination half-life of 2-6 hours. The usual starting dose of metformin is 500 mg twice a day or 850 mg once a day given with meals with maximum of 2550 mg per day. Such conventional multiple dose therapy may lead to substantial fluctuation in the plasma concentration of the drug, especially in chronic administration. A dosage form, which allows the drug to be released over an extended period of time in a rate-controlled manner i.e. a controlled release metformin dosage form, leads to a constant therapy and thereby eliminating the multiple dosing and side effects.

The prior art enumerates several attempts to prepare a controlled release metformin composition. U. S. Pat. No. 5,955,106 (hereinafter referred to as '106 patent) disclosed a pharmaceutical composition comprising metformin and a hydrocolloid forming retarding agent with the residual moisture content of about 0.5-3%. The said moisture level was maintained such that usual capping problem associated with the high-dose tablet formulations is avoided. The hydrocolloid forming retarding agents used in this invention, were selected from a group of cellulose derivatives, dextrins, starches, carbohydrate polymers, natural gums, xanthane, alginates, gelatin, polyacrylic acid, polyvinyl alcohol and polyvinyl pyrollidones. The matrix tablets so formed could optionally be film coated with polymers such as soluble cellulose derivatives, ethyl cellulose, poly(ethacrylate-methyl methacrylate) dispersion and plasticizers such as diethyl phthalate and macrogol to mask the taste or to additionally retard the release. Metformin has a comparatively large dose and thus the dosage form tends to become bulky and difficult to swallow. This coupled with the problem of poor compressibility of metformin reduces the flexibility in formulating the composition to obtain the optimum release profile. We have found a dosage form comprising metformin or its pharmaceutically acceptable salt, two or more swellable polymers wherein at least one polymer is anionic, one or more pharmaceutically acceptable excipient(s) that improve the compressibility of the core composition and optionally a coat comprising one or more water insoluble polymer(s) surrounding the core wherein the composition enables the poor compressibility of metformin to be brought under control and allows convenient modulation of release without necessarily requiring moisture control in the range of 0.5-3%.

PCT application Number WO 9947128 disclosed a pharmaceutical composition wherein particles of an inner phase comprising a highly water soluble active ingredient and an extended release material are dispersed in an outer solid continuous phase comprising an extended release material. In the system of the present invention however, the inner phase is in the form of a core, which is surrounded by a water insoluble polymeric coat. The system of WO 9947128 consists of a matrix of an inner phase in an outer phase and would be considered as a “matrix-system” by those skilled in the art. The system of the present invention has two distinct components viz. a core and a coat such systems are regarded as reservoir type systems and provide a distinctly different kinetics and mechanisms of release, alternately, the present invention has optionally no coat, thus is a single phase matrix system without coat and is thus different.

U.S. Pat. No. 6,099,859 disclosed an osmotic controlled release tablet composition. Osmotic controlled release system differs from the present invention in that the release of a drug occurs through an orifice in the semipermeable membrane coating and the osmotic pressure within the system exercises control on the release.

The prior art does not disclose a dosage form for treatment of diabetes mellitus and conditions associated with it comprising a compressible controlled release core composition comprising metformin or its pharmaceutically acceptable salt, two or more swellable polymers wherein at least one polymer is an anionic polymer, one or more pharmaceutically acceptable excipient(s) that improve the compressibility of the core composition and optionally a coat comprising one or more water insoluble polymer(s) surrounding the core.

c) Combinations of Biguanide and Sulfonyl Ureas:

The sulfonyl ureas used in the treatment of diabetes mellitus type 2 include acetohexamide, carbutamide, chlorpropamide, glipizide, glyburide (glibenclamide), glimepiride, gliclazide, glibomuride, gliquidone, glisoxepid, glyhexamide, phenbutarnide, tolazamide, tolbutamide, tolcyclamide, etc. These sulfonyl ureas are used as their bases and not as salts. The mechanism of action of these drugs involves lowering of blood glucose concentration mainly by stimulating release of endogenous insulin from beta cells of the panrreas, and thus they act as hypoglycemic agents. The sulfonyl ureas are used as an adjunct to diet for the management of non-insulin dependent diabetes mellitus in patients whose hyperglycemia cannot be controlled by diet alone. To achieve maximum reduction in post-prandial blood-glucose concentration, the sulfonyl urea is administered 30 minutes prior to each meal. Out of these long-acting sulfonyl ureas can be enumerated as carbutamide, chlorpropamide, glibenclamide, gliclazide, glimepiride.

In particular, glimepiride has a more pronounced in vitro insulin secretory activity as compared to the other sulfonyl ureas. The drug achieves therapeutically equivalent blood glucose control with lower fasting plasma insulin levels. Since hyperinsulinaemia leads to acceleration of atherosclerosis, glimepiride has a major advantage over currently available sulfonyl ureas. In addition, extrapancreatic effects may also play a role in the activity of glimepiride. This is supported by both preclinical and clinical studies demonstrating that glimepiride administration can lead to increased sensitivity of peripheral tissues to insulin. It is effective in controlling blood glucose without deleterious changes in the plasma lipoprotein profiles of NIDDM patients. Glimepiride is also safe for use in the elderly and those with renal impairment. It has a quick onset of action and, at the same time, its duration of action is prolonged so that it needs to be administered only once a day. Hence, glimepiride is the preferred sulfonyl urea.

As referred to herein, ‘conditions associated with diabetes mellitus’ includes those conditions associated with the pre-diabetic state, conditions associated with diabetes mellitus itself and complications associated with diabetes mellitus. When used herein the term ‘conditions associated with pre-diabetic state’ includes conditions such as insulin resistance, including hereditary insulin resistance, impaired glucose tolerance, obesity and hyperinsulinaemia. ‘Conditions associated with diabetes mellitus’ itself include hyperglycemia, insulin resistance, including acquired insulin resistance and obesity. Further conditions associated with diabetes mellitus itself include hypertension and cardiovascular disease, especially atherosclerosis and conditions associated with insulin resistance. Conditions associated with insulin resistance include polycystic ovarian syndrome and steroid induced insulin resistance and gestational diabetes. ‘Complications associated with diabetes mellitus’ includes renal disease, especially renal disease associated with Type II diabetes, neuropathy and retinopathy. Renal diseases associated with Type II diabetes include nephropathy, glomerulonephritis, glomerular sclerosis, nephritic syndrome, hypertensive nephrosclerosis and end stage renal disease.

The 54^{th} edition of the Physicians' Desk Reference, copyright 2000, suggests that the monotherapy with metformin hydrochloride, commercially available under the trade name Glucophage® from Bristol-Myers Squibb Co., may be effective in patients who have not responded to sulfonyl ureas or who have only a partial response to sulfonyl ureas or who have ceased to respond to sulfonyl ureas. In such patients, if adequate glycemic control is not attained with Glucophage® monotherapy, the combination of Glucophage® and a sulfonyl urea may have a synergistic effect. Also, monotherapy with the sulfonyl ureas has been found to give a positive response, which lasts for 4-5 years, but it becomes ineffective in a large number of patients over a period of time. This is referred to as the “secondary failure” associated with the oral therapy with hypoglycemic agents. In both these cases, a combination of biguanides and sulfonyl ureas is used. The biguanides are able to act on insulin resistance but cannot stimulate insulin secretion, while the sulfonyl ureas can stimulate insulin release but are unable to act on insulin resistance. A combination therapy of a biguanide and a sulfonyl urea has a synergistic effect on glucose control, since both agents act by different but complementary mechanisms. Although the 54^{th} Edition of Physician Desk Reference is suggesting of a combination therapy of a biguanide and a sulfonyl ureas it does not disclose the manner of delivering them from a single unit dosage form.

Particularly it does not disclose immediate release of a long-acting sulfonyl urea for maximum reduction of post-prandial glucose and sustained release of a biguanide as a once-a-day dosage regimen.

U.S. Pat. No. 5,922,769 ('769) claims a method of treating non-insulin dependent diabetes mellitus in cases of secondary failure comprising administering to the subject in need of the same a combination of glibenclamide and metformin, expressed as the hydrochloride, in a weight ratio higher than 1:100. The patent also discloses the results of a clinical study, which indicates that the maximum dose of glibenclamide, which does not cause any side-effects, is 15 mg/day, while that for metformin is 1500 mg/day, and that the use of such a combination in a ratio lower than that claimed would result in formulations that do not attain the optimum therapeutic effect. The patent claims the combination of glibenclamide and metformin in a tablet form. The patent does not disclose a formulation wherein the sulfonyl urea will be released immediately and the biguanide will be released in a controlled manner.

U.S. Pat. No. 6,031,004 ('004) discloses the use of a combination of novel salts of metformin and glyburide, in the treatment of diabetes mellitus type 2. In this invention, both metformin salt and glyburide are released immediately.

U.S. Pat. No. 6,099,862 ('862) claims a controlled release pharmaceutical tablet which consists essentially of (a) a core consisting essentially of: (i) metformin or a pharmaceutically acceptable salt thereof, (ii) glipizide, (iii) polyvinylpyrrolidone, and (iv) sodium lauryl sulfate, (b) optionally a seal coat around the core, (c) a semipermeable membrane coating covering said core comprising—(i) cellulose acetate, (ii) polyethylene glycol with an average molecular weight between 380 and 420, and (iii) a plasticizer, and (d) at least one passageway in the semipermeable membrane to allow the release of the metformin and glipizide from the core to the environment of use to provide therapeutic levels of metformin and glipizide from twelve to twenty-four hour periods. A dosage form comprising a biguanide and a long-acting sulfonyl urea that immediately releases a sulfonyl urea, such as glimepiride, after administration of the dosage form, and releases, a biguanide, such as metformin, such that the biguanide is released in a controlled manner is not disclosed by the '862 patent.

The prior art thus does not mention any such formulations or systems containing combinations of a biguanide and long-acting sulfonyl urea wherein the long-acting sulfonyl urea is released immediately and the biguanide is released in a controlled manner.

### OBJECT OF THE INVENTION

The object of the present invention is to provide a dosage form for treatment of diabetes mellitus and conditions associated with it, wherein the dosage form comprises metformin or its pharmaceutically acceptable salt in a readily compressible and controlled release form.

The further object of the present invention is to provide a dosage form for treatment of diabetes mellitus and conditions associated with it, comprising a long-acting sulfonyl urea and a biguanide wherein the long-acting sulfonyl urea is released immediately and the biguanide is released in a controlled manner.

### SUMMARY OF THE INVENTION

The present invention provides a dosage form for the treatment of diabetes mellitus and conditions associated with it, comprising a compressible controlled release core composition comprising metformin or its pharmaceutically acceptable salt, two or more swellable polymers wherein at least one polymer is an anionic polymer, one or more pharmaceutically acceptable excipient(s) that improve the compressibility of the core composition and optionally a coat comprising one or more water insoluble polymer(s) surrounding the core.

The present invention further provides a dosage form for the treatment of diabetes mellitus and conditions associated with it comprising an immediate release composition comprising a long-acting sulfonyl urea and a controlled release composition comprising a biguanide.

### BRIEF DESCRIPTION OF THE DRAWINGS

FIG. 1 shows the plasma concentration vs time profile obtained upon administration of one embodiment of the controlled release metformin dosage form as exemplified in Example 1 in comparison to Glucophage XR of the present invention. FIG. 2 shows the plasma concentration vs time profile obtained upon administration of one embodiment of the controlled release metformin dosage form as exemplified in Example 2 in comparison to Glucophage XR.

### DETAILED DESCRIPTION OF THE INVENTION

The dosage form of the present invention is designed to allow a controlled release of the biguanide.

Examples of biguanides that may be used in the present invention include metformin, phenformin and buformin and their pharmaceutically acceptable salts.

The present invention particularly provides a dosage form for the treatment of diabetes mellitus and conditions associated with it, comprising a compressible controlled release core composition comprising metformin or its pharmaceutically acceptable salt, two or more swellable polymers wherein at least one polymer is an anionic polymer, one or more pharmaceutically acceptable excipient(s) that improve the compressibility of the core composition and optionally a coat comprising one or more water insoluble polymer(s) surrounding the core.

In preferred embodiments of the present invention the amount of metformin or its pharmaceutically acceptable salts is about 500 mg to about 1000 mg.

The swellable polymeric materials used in the present invention are hydrogels that swell in, and retain a significant amount of water. These swellable polymers are polymeric hydrogels (crosslinked or uncrosslinked) which swell or expand significantly in water, usually exhibiting a 2 to 50 fold or greater volume increase. The crosslinked polymers will swell and will not dissolve; uncrosslinked polymers may dissolve subsequent to swelling although dissolution is not a necessary consequence.

Examples of the swellable polymers that can be used in the present invention include:

a cellulose and cellulose derivatives such as methylcellulose (MC), ethylcellulose (EC), hydroxyethylcellulose (HEC), hydroxypropyl cellulose (HPC), hydroxypropyl methylcellulose (HPMC), hydroxypropyl ethylcellulose (HPEC), carboxymethyl cellulose (CMC), crosslinked carboxymethyl cellulose (croscarmellose) and its alkali salts, ethylhydroxyethylcellulose (EHEC), hydroxyethyl methylcellulose (HEMC), hydrophobically modified hydroxyethyl cellulose (HMHEC), hydrophobically modified ethylhydroxyethylcellulose (HMEHEC), carboxymethyl hydroxyethylcellulose (CMHEC), carboxymethyl hydrophobically modified hydroxyethyl cellulose (CMHMHEC) and the like;

alkylene oxide homopolymers such as polypropylene oxide, preferably ethylene oxide homopolymers and the like;

disintegrant such as cross-linked polyvinylpyrrolidone, cross-linked sodium carboxymethylcellulose, carboxymethyl starch, sodium carboxymethyl starch, potassium methacrylate-divinylbenzene copolymer, polyvinyl alcohols, amylose, cross-linked amylose, pregelatinized starch, starch and starch derivatives.

gums of plant, animal, mineral or synthetic origin such as (i) agar, alginates, carrageenan, furcellaran derived from marine plants, (ii) guar gum, gum arabic, gum tragacanth, karaya gum, locust bean gum, pectin derived from terrestrial plants, (iii) microbial polysaccharides such as dextran, gellan gum, rhamsan gum, welan gum, (iv) synthetic or semi-synthetic gums such as hydroxypropyl guar and the like;

It is also possible to use vinyl pyrrolidone polymers or polyvinylpyrrolidone (PVP), also referred to as Povidone as the swellable polymers. These are the synthetic polymers consisting essentially of linear 1-vinyl-2-pyrrolidinone groups, the degree of polymerization of which results in polymers of various molecular weights, the molecular weight ranging between 2500 and 3,000,000 Daltons. PVP is commercially available as Kollidone® (BASF), Plasdone® and Peristone® (General Aniline). PVP is classified into different grades on the basis of its viscosity in aqueous solution. Different grades of PVP available are PVP K-12, PVP K-15, PVP K-17, PVP K-25, PVP K-30, PVP K-60, PVP K-90 and PVP K-120. The K-value referred to in the above nomenclature is calculated from the viscosity of the PVP in aqueous solution, relative to that of water. The preferred vinyl pyrrolidone polymer used as a swellable polymer is PVP K-30, having an approximate molecular weight of 50000 Daltons.

The anionic swellable polymers used in the present invention are selected from the group consisting of homo-polymers and copolymers of polyacrylic acid and polyacrylic acid derivatives, various starch derivatives, cellulose derivatives, gums and the like.

Homopolymers of polyacrylic acid and its derivatives that can be used in the present invention, include, but are not limited to, various grades sold under the trade name of Carbopol® by BFGoodrich. These are high molecular weight, crosslinked, acrylic acid-based polymers. Carbopol homopolymers are polymers of acrylic acid crosslinked with allyl sucrose or allylpentaerythritol. Carbopol copolymers are polymers of acrylic acid, modified by long chain (C₁₀-C₃₀) alkyl acrylates and crosslinked with allylpentaerythritol. The USP-NF, British Pharmacopoeia have adopted the generic name “Carbomer” for various Carbopol® Homopolymer polymers. The Japanese Pharmaceutical Excipients list carbopol homopolymers as “carboxyvinyl polymer” and “carboxy polymethylene”. A range of different pharmaceutical grade polymers, each with special properties and applications depending upon the viscosity of the polymers and the applicability are available. The Carbopol grades 934 NF, 2984, 5984 EP, for example can be used for stable emulsions and suspensions for water and solvent-based gels having viscosity ranging from 30,500 to 45,000 cps for 0.5% solution at pH 7.5. Carbopol 934P NF, 974P NF having viscosity ranging from 29,400 to 39,400 cps can be used especially for oral and mucoadhesive applications such as controlled release tablets and oral suspensions. The grades 940 NF, 980 NF having viscosity ranging from 40,000 to 65,000 cps are to be used for topical gels. The low viscosity grades of Carbopol namely, 941NF, 981 NF can be used for low viscosity sparkling clear topical gels. The carbopol 934P is high purity grade of Carbopol 934. Depending on drug solubility, drug hydrophilicity and basic strength, polymer concentration and test medium pH, Carbopol 934 P can show zero-order release profiles in tableting applications.

It is also possible to use in the present invention, co-polymers of acrylate or methacrylate monomers, for example polymethacrylates marketed under the brand names of Eudragit® as the anionic swellable polymers. Eudragit L and S also referred as methacrylic acid copolymers are the copolymerization products of methacrylic acid and methyl methacrylate. The ratio of free carboxy groups to the ester is approximately 1:1 in Eudragit L and approximately 1:2 in Eudragit S. Eudragit® RS and RL, also referred to as ammoniomethacrylate copolymers are copolymers synthesized from acrylic acid and methacrylic acid esters with Eudragit® RL type having 10% of functional quaternary ammonium groups and Eudragit® RS having 5% of functional quaternary ammonium groups.

Examples of starch derivatives that can be used as the anionic polymers include, but are not limited to, sodium starch glycolate and various other anionic starch derivatives and the like.

Examples of other anionic polymers that can be used as the swellable polymers in the present invention, include, but are not limited to gums such as sodium alginate, sold under the name of KELTONE® , propylene glycol alginate and the like. Particularly, the preferred anionic polymer used in the present invention is xanthan gum, which is a high molecular weight polysaccharide, available in various grades, viscosity ranges and of different particle sizes. The xanthan gum used in the present invention is the food fine (FF) grade, 200 mesh, supplied by Jungbunzlauer.

Examples of anionic cellulose derivatives that can be used as the swellable polymers in the present invention, include, but are not limited to sodium carboxymethyl cellulose, potassium carboxymethyl cellulose, calcium carboxymethyl cellulose, cross linked sodium carboxymethyl cellulose known as crosscarmellose, sold under the name of Ac-Di-SOL®, cellulose acetate phthalate, hydroxypropyl methyl cellulose phthalate and the like.

Particularly, the preferred swellable polymers used in the present invention are the cellulose ethers. Cellulose ethers are nonionic polymers however some cellulose ethers may be anionic for example cellulose ethers with some hydroxyl groups esterified for example hydroxypropyl methyl cellulose phthalate, cellulose acetate phthalate or cellulose ethers with the hydroxyl group further reacted to incorporate an anionic functional group for example carboxymethyl cellulose calcium and the like. The examples of the cellulose ethers include methylcellulose (MC), ethylcellulose (EC), hydroxyethylcellulose (HEC), hydroxypropyl cellulose (HPC), hydroxypropyl methylcellulose (HPMC), hydroxypropyl ethylcellulose (HPEC), carboxymethyl cellulose (CMC), crosslinked carboxymethyl cellulose (croscarmellose) and its alkali salts, ethylhydroxyethylcellulose (EHEC), hydroxyethyl methylcellulose (HEMC), hydrophobically modified hydroxyethyl cellulose (HMHEC), hydrophobically modified ethylhydroxyethylcellulose (HMEHEC), carboxymethyl hydroxyethylcellulose (CMHEC), carboxymethyl hydrophobically modified hydroxyethyl cellulose (CMHMHEC) and the like.

More preferably, the swellable polymers used in the present invention include various grades of the hydroxypropylmethyl cellulose available under the brand Name of Methocel. The grades available are categorized depending upon the chemical substitution and hydration rates. The Methocel grade K, for example is used for HPMC having % methoxy content of 19-24% and hydroxypropyl content of 7-12% with a fastest relative rate of hydration. Similarly, the Methocel E grade is used for 28-30% methoxy content and 7-12% of hydroxypropyl with a more faster relative hydration rate as compared to the K grade. The F grade of Methocel indicates a 27-30% methoxy content and 4-7.5% of hydroxypropyl content with a slow relative hydration rate. The Methocel Grade A indicates a 27.5-31.5% methoxy content and 0% hydroxypropyl with slowest rate of hydration.

HPMC is further categorized based on the particle size. For example, the premium grades of Methocel have particle size in the range such that 100% particles are less than 30 mesh screen and 99% of the particles are less than 40 mesh screen. The E grade has a particle size in the range such that 95% particles are less than 100 mesh screen whereas the K series has 90% of particles less than 100 mesh screen.

HPMC is further characterized according to viscosity exhibited by the 2% HPMC solution in water. The Methocel grades based on the viscosities are K100LVP, K4M, K15MP, K100MP and E4MP. The K100LVP indicates a minimal viscosity of 100 cps, K4M of 4000 cps, K15MP of 15,000 and K100MP of 100,000 and several low viscosity grades such as E3, E5, E6, E15, E50 and K3.

In one embodiment of the present invention, the swellable polymer, which is a high viscosity grade cellulose derivative, preferably hydroxypropyl methyl cellulose, commercially available as Methocel® K100M, with a 2% w/w aqueous solution of HPMC having a viscosity in the range from about 80,000 to about 120,000 cps units is used. In a particularly preferred embodiment the swellable polymer is a mixture of high viscosity grade of HPMC having a viscosity greater than about 10,000 cps and a low viscosity grade HPMC having a viscosity equal to or less than about 10,000 cps. In one preferred embodiment of the present invention, one of the low viscosity grades of the HPMC used has viscosity of about 4000 cps and is commercially available as Methocel® K4M, and the other high viscosity grade of HPMC used has a viscosity of about 100,000 cps, and is commercially available as Methocel® K100M.

In preferred embodiments of the present invention the anionic polymers is selected from a group of polyacrylic acid or a xanthan gum or mixtures thereof. In one preferred embodiments of the present invention, Carbopol 934P is used as one of the anionic swellable polymers.

In the particularly preferred embodiments of the present invention, a combination of a high molecular weight HPMC and Carbopol 934P is used. HPMC may be used in the concentration ranging from about 10 to 15% w/w of the total weight of the tablet. Amounts of Carbopol 934P that can be used in preferred embodiments of the present invention may vary from about 5 to about 20% w/w of the total weight of the tablet.

In the still further preferred embodiments of the present invention, a combination of high molecular weight HPMC and Xanthan gum Type FF is used. HPMC may be used in the concentration ranging from about 10 to 20% w/w of the total weight of the tablet. More preferably, the HPMC used is a mixture of high viscosity grade of HPMC having a viscosity greater than about 10,000 cps and a low viscosity grade HPMC having a viscosity equal to or less than about 10,000 cps. In one preferred embodiment of the present invention, one of the low viscosity grades of the HPMC used has viscosity of about 4000 cps and is commercially available as Methocel® K4M, and the other high viscosity grade of HPMC used has a viscosity of about 100,000 cps, and is commercially available as Methocel® K100M. Amounts of xanthan gum that can be used in preferred embodiments of the present invention may vary from about 5 to about 20% w/w of the total weight of the tablet.

Amounts of the swellable polymers that can be used in preferred embodiments of the present invention may range from about 15 to about 40% of the total weight of the tablet.

The dosage form of the present invention contains one or more pharmaceutically acceptable excipient(s) that improve the compressibility of the core composition. These excipient(s) allow a wider range of moisture than the range of 0.5 to 3% w/w to be retained in the core while enabling the poor compressibility of metformin to be brought under control. The inventors do not subscribe to any theory but perhaps these excipient(s) by their wicking or dessicant action for retaining moisture or by their compressibility characteristics enable the compressibility to be brought under control. Thus excipient(s) that further modulate the release of metformin from the core can be included in the composition and provide flexibility to the formulation in obtaining the desired release profile.

Examples of the excipient(s) that improve the compressibility of the core composition include microcrystalline cellulose, powdered cellulose, silicified microcrystalline cellulose, dextrins and dextrans, colloidal silicon dioxide, kaolin, titanium dioxide, fused silicon dioxide, alumina, bentonite, magnesium silicate, magnesium trisilicate, anhydrous calcium sulfate, magnesium aluminium silicate and the like.

In preferred embodiments of the present invention, the excipient used to improve the compressibility of the core composition is microcystaline cellulose.

The dosage form of the present invention may optionally contain the excipient(s) that further modulate the rate of release of metformin from the core. These excipient(s) are selected from the group consisting of osmotic agents, inorganic or organic weak acids or weak bases and surfactants.

Examples of the osmogent(s) used in the present invention include all pharmaceutically acceptable and pharmacologically inert water-soluble compounds referred to in the pharmacopoeias such as United States Pharmacopoeia, as well as in Remington: The Science and Practice of Pharmacy; edition 19; Mack Publishing Company, Easton, Pa. (1995). Pharmaceutically acceptable water-soluble salts of inorganic or organic acids, or non-ionic organic compounds with high water solubility, e.g. carbohydrates such as sugar, or amino acids, are generally preferred. The examples of agents used for inducing osmosis include inorganic salts such as magnesium chloride or magnesium sulfate, lithium, sodium or potassium chloride, lithium, sodium or potassium hydrogen phosphate, lithium, sodium or potassium dihydrogen phosphate, salts of organic acids such as sodium or potassium acetate, magnesium succinate, sodium benzoate, sodium citrate or sodium ascorbate; sodium carbonate or sodium bicarbonate; carbohydrates such as mannitol, sorbitol, arabinose, ribose, xylose, glucose, fructose, mannose, galactose, sucrose, maltose, lactose, raffinose, inositol, xylitol, maltitol; water-soluble amino acids such as glycine, leucine, alanine, or methionine; urea and the like, and mixtures thereof.

Examples of inorganic or organic weak acids or weak bases used in the present invention include, but are not limited to citric acid, lactic acid, ascorbic acid, tartaric acid, malic acid, fumaric acid and succinic acid and salts thereof.

Examples of the surfactants used in the present invention include, but are not limited to glyceride; partial glycerides, glyceride derivatives, polyhydric alcohol esters, PEG and PPG esters, polyoxyethylene and polypropylene sorbitan esters; sodium lauryl sulfate and the like.

Preferred embodiments of the dosage form of the present invention include sodium chloride as the osmogent and sodium bicarbonate as the weak base.

The additional excipient(s) used in the dosage form of the present invention include those excipient(s) which are generally used during the preparation of granules, compression e.g lubricants, glidants, disintegrants and the like. These excipients are discussed in ‘Remington's Pharmaceutical Sciences, 18^{th} edition, page 1635-38(1990).

The dosage form of the present invention has optionally a coat comprising one or more water insoluble polymer(s) surrounding the core. Examples of water insoluble polymers that may be used include cellulose ether derivatives, acrylic resins, copolymers of acrylic acid and methacrylic acid esters. Combined with the polymer material may be a hydrophobic agent, which may be a fatty acid of 10 or more carbon atoms, wax or the salts of a fatty acid or 10 or more carbon atoms such as magnesium stearate or calcium stearate. The particular hydrophobic agent may be a mixture of stearates, which contain other fatty acids because the product is derived from a natural source. The purpose of the hydrophobic agent is to reduce the permeability of the water insoluble, water permeable polymer to water by adding from 25% to 50% by weight of the hydrophobic agent to said polymer based on the total combined weight of the hydrophobic agent and said polymer. Small amounts of stearates will reduce tackiness and very large amounts will reduce water permeability.

In the most preferred embodiments of the present invention, the water insoluble polymeric material used to coat the core is a highly water permeable, pH independent methacrylic-methacrylate polymer with quaternary ammonium groups.

Plasticizers may be added to the water insoluble polymer to control any brittleness in the polymeric coat. The plasticizer used in the present invention may be selected from the group consisting of diethyl phthalate, diethyl sebacate, triethyl citrate, diethyl phthalate, glycerol, sorbitol, crotonic acid, propylene glycol, castor oil, citric acid esters, dibutyl phthalate, dibutyl sebacate, and mixtures thereof.

The amount of the coating applied on the core may vary from about 0.5 to about 20% w/w of the total weight of the dosage form, depending on the core composition. In the preferred embodiments the amount of coating applied is from about 3% to about 5% by weight of the total weight of the dosage form.

The mixture of metformin or its pharmaceutically acceptable salt, two or more swellable polymers wherein at least one polymer is an anionic polymer, one or more pharmaceutically acceptable excipient(s) that improve the compressibility of the core composition and optionally excipient(s) that modulate the release of metformin from the core is converted into a core by conventional means. These include wet granulation, dry granulation, direct compression, pelletization, extrusion-spheronization, layering onto inert particles such as non-pareil seeds, and the like. The core may be further modified by compression in a tablet press. The core so obtained may be in the form of granules, pellets or tablets. The tablets may be single layered or compressed into multilayered tablets such as bilayered tablets. In the case of dry granulation, the dry mixture of metformin or its pharmaceutically acceptable salt, swellable polymer(s), optionally excipient(s) that modulate the release of metformin from the core and the excipients that improve the compressibility of the core composition is compressed to obtain slugs, which are then passed through suitable sieves to obtain granules. In wet granulation, the mixture is granulated with a liquid preferably, a mixture of isopropyl alcohol and water. The dried granules are compressed on a tablet compression machine. In case of direct compression, the components of the system are mixed thoroughly and directly compressed on a tablet compression machine.

The compressed cores, obtained by any one of the above methods, may optionally be subjected to coating, moulding, spraying, or immersion using a coating solution comprising one or more water insoluble polymer(s) to form the water insoluble polymeric coat. The water insoluble polymer(s) and other adjuvants such as plasticizers, opacifiers, pigments and the like are dissolved or dispersed in a suitable organic or aqueous solvent to form the coating solution.

The present invention also provides a novel dosage form for the treatment of diabetes mellitus and conditions associated with it comprising an immediate release composition comprising a long-acting sulfonyl urea and the controlled release composition comprising the biguanide.

Those skilled in the art would realize that by immediate release of the long-acting sulfonyl urea typically one means that release of the long-acting sulfonyl urea occurs substantially immediately after administration of the dosage form, for instance or for illustrative purpose, about 80% or more of the total amount of sulfonyl urea may be released in 30 minutes.

Examples of sulfonyl ureas that may be used in the present invention include carbutamide, chlorpropamide, glyburide (glibenclamide), glimepiride, gliclazide, glibomuride, glyhexamide, phenbutamide, tolazamide, tolbutamide, tolcyclamide and the like.

In one embodiment of the present invention, the dosage form for the treatment of diabetes mellitus and conditions associated with it comprises a long-acting sulfonyl urea immediate release composition and a compressible controlled release core composition comprising metformin or its pharmaceutically acceptable salt, two or more swellable polymers wherein at least one polymer is an anionic polymer, one or more pharmaceutically acceptable excipient(s) that improve the compressibility of the core composition and optionally a coat comprising one or more water insoluble polymer(s) surrounding the core. The preferred long-acting sulfonyl urea of the present invention is glimepiride.

The invention covers any dosage form in which the immediate release composition comprising a long-acting sulfonyl urea and the controlled release composition comprising the biguanide are physically separated, or compartmentalized, so as to achieve different release rates of the two drugs. Such separation, or compartmentalization, may be on a macro-scale, for instance, with the different drugs being incorporated into separate units (such as tablets, powder, granules, pellets etc) for simultaneous or sequential administration, or separation of the two drugs may be on a micro-scale, for instance, with the two drugs being present within the same unit. Two separate units when present are formed into a single unit dosage form by filling them into capsules.

In the dosage form of the present invention, the immediate release long-acting sulfonyl urea composition and the controlled release biguanide composition, may be in the form of either multiparticulates such as particles, pellets or granules, or present as concentric or laminar tablet layers or as single units such as a compressed tablet. The multiparticulates may be made by any of the conventional methods, including mixing, granulation, extrusion, spheronisation, layering of non-pareil seeds, etc, and various other methods known to a person skilled in the art. A compressed tablet core may be obtained by compressing the mutiparticulates in a tablet die. The biguanide composition may be surrounded by a controlled release coating comprising controlled release material selected from the group consisting of enteric polymers, water insoluble polymers, hydrophobic compounds, hydrophilic non-polymeric compounds, hydrophilic polymers and the like, using conventional coating methods. The coated multiparticulates or tablets of the biguanide composition and the uncoated multiparticulates or tablets of the second long-acting sulfonyl urea composition, may be filled into capsules. Alternatively, tablets of the biguanide composition may be surrounded by the immediate release long-acting sulfonyl urea composition and compressed in a compression coating tablet machine or a second layer of the long-acting sulfonyl urea composition may be compressed onto the compressed biguanide composition to form bilayer tablets.

In one embodiment of the present invention, the immediate release long-acting sulfonyl urea composition is introduced by mixing the sulfonyl urea with pharmaceutical adjuvants such as film-forming agents, plasticisers and the like, in a suitable solvent or solvent system, and coating the controlled release biguanide core composition, using conventional coating methods known to a person skilled in the art. Examples of film-forming agents that may be used in the present invention along with the long-acting sulfonyl urea include cellulose derivatives such as cellulose acetate phthalate, hydroxypropyl methylcellulose, hydroxypropyl cellulose, hydroxypropyl ethylcellulose, ethyl cellulose, methyl cellulose, methacrylic acid/methacrylate esters, polyvinyl acetate phthalate, shellac and the like, or mixtures thereof. Hydroxypropyl methylcellulose (HPMC) is used as the preferred film-forming agent along with the long-acting sulfonyl urea in the present invention, in an amount ranging from about 2% to about 20% by weight of the coated biguanide-containing core, more preferably from about 15% to about 20% by weight of the core. Examples of plasticisers that may be used in the present invention include, but are not limited to glycerol, propylene glycol, polyethylene glycol, sorbitol, triacetin, diethyl phthalate, mineral oil, petrolatum, lanolin and the like. In a preferred embodiment of the present invention, polyethylene glycol (PEG) 6000 is used as the plasticiser in an amount ranging from 0% to about 5% by weight of the core, more preferably from about 0.1% to about 1% by weight of the core. The long-acting sulfonyl urea is dissolved in methylene chloride. HPMC dispersed in isopropyl alcohol and the long-acting sulfonyl urea are mixed in a solvent system and further mixed with the PEG 6000 previously melted and dissolved in water. The solution thus obtained is used to coat the controlled release biguanide core composition to a desired weight gain, in a conventional tablet-coating pan. The tablets are then dried in a tray drier at a temperature of 40-50° C. for 24 hours.

In preferred embodiments of the present invention the dosage form comprises 1.0 mg of glimepiride and 500.0 mg of metformin or its pharmaceutically acceptable salt.

The examples that follow do not limit the scope of the invention and are presented as illustrations.

### EXAMPLE 1

One of the embodiments of the present invention is described in the following example. The controlled release tablets of this embodiment are prepared as per the formula mentioned in Table 1 below.

Stage A: Metformin hydrochloride and sodium chloride were milled and passed through 100 mesh sieve. Carbopol 934P, hydroxypropyhnethyl cellulose, microcrystalline cellulose and starch were passed through 60 mesh sieve and further uniformly mixed with the blend of metformin and sodium chloride.

Stage B: The powder mixture was granulated with polyvinyl pyrrolidone K-30 dissolved in isopropyl alcohol and water mixture. The granules were dried at 45° C. The dried granules were passed through a mill.

Stage C: Talc, magnesium stearate and colloidal silicon dioxide were passed through 60 mesh sieve and then mixed with the granules. The lubricated granules were compressed at the required tablet weight.

Stage D & E: The compressed cores were further coated with a polymeric dispersion comprising Eudragit RL100 in isopropyl alcohol and acetone containing triethyl citrate, talcum and titanium dioxide.

TABLE 1

| | | | % w/w |
|---|---|---|---|
| Stage | | Quantity per | of the |
| Number | Ingredients (mg) | dosage form (mg) | tablet |
| Stage A | | | |
| 1. | Metformin HCL (100#) | 500.0 | 47.619 |
| 2. | Carbopol 934P | 100.0 | 9.523 |
| 3. | Sodium chloride | 40.0 | 3.809 |
| 4. | HPMC K100M | 180.0 | 17.143 |
| 5. | Microcrystalline cellulose | 50.0 | 4.7619 |
| 6. | Starch | 75.0 | 7.1428 |
| Stage B | | | |
| 1. | Polyvinylpyrrolidone (K-30) | 20.0 | 1.904 |
| 2. | Isopropyl alcohol | Quantity sufficient | q.s |
| 3. | Purified Water | Quantity sufficient | q.s |
| Stage C | | | |
| 1. | Talc | 20.0 | 1.904 |
| 2. | Magnesium stearate | 10.0 | 0.952 |
| 3. | Colloidal Silicon dioxide | 10.0 | 0.952 |
| | Total | 1000.0 | 95.23 |
| Stage D | | | |
| 1. | Eudragit RL 100 | 37.04 | 3.527 |
| 2. | Isopropyl alcohol | Quantity sufficient | q.s |
| 3. | Acetone | Quantity sufficient | q.s |
| 4. | Triethyl citrate | 3.70 | 0.352 |
| Stage-E | | | |
| 1. | Talcum | 5.56 | 0.529 |
| 2. | Titanium dioxide | 3.70 | 0.352 |
| 3. | Isopropyl alcohol | Quantity sufficient | q.s |

| | | |
|---|---|---|
| Total weight of the coated tablet | 1050 | 100 |

The tablets so obtained were subjected to dissolution testing using USP type I dissolution apparatus. The dissolution medium used was 900 ml of 0.1N HCl for 0-2 hours and 900 ml of simulated intestinal fluid, pH 6.8, for 2-12 hours. The results of the dissolution test are mentioned in Table 2 below.

TABLE 2

| Time (hours) | % drug released (±SD) |
|---|---|
| 1 | 12.00 ± 1.53 |
| 2 | 29.08 ± 0.37 |
| 4 | 47.82 ± 0.28 |
| 8 | 69.20 ± 0.82 |
| 12 | 84.88 ± 3.42 |

### EXAMPLE 2

This example describes another embodiment of the present invention. The tablets were prepared according to the formula described in Table 3.

Stage A: Metformin Hydrochloride was milled and passed through 100 mesh. Carbopol 934P and sodium bicarbonate were passed through 60 mesh and all the ingredients were mixed thoroughly.

Stage B: The blend was granulated with PVP K-30 dissolved in isopropyl alcohol. The wet mass was passed through 20 mesh and the wet granules were dried in Fluidized Bed dryer at 45° C. The dried granules were passed through 30 mesh.

Stage C: All the ingredients of Stage C were passed through 60 mesh and mixed with the dried, sifted granules of metformin hydrochloride.

Stage D: This mixture was further granulated with PVP K-30 in isopropyl alcohol. The wet mass was passed through 20 mesh. The granules so obtained were dried in the Fluidized Bed dryer at 45° C.

Stage E: All the ingredients of the step E were passed through 60 mesh. This mixture was used to lubricate the dried granules. The lubricated granules were compressed into tablets of 20.5×9.0 mm capsule shaped punch.

Stage P & G: The compressed cores were further coated with a polymeric dispersion comprising Eudragit RL100 in isopropyl alcohol and acetone containing triethyl citrate, talcum and titanium dioxide.

TABLE 3

| Stage | | Quantity per dosage | % w/w of |
|---|---|---|---|
| Number | Ingredients (mg) | form (mg) | the tablet |
| Stage A | | | |
| 1. | Metformin HCL (100#) | 500.0 | 50.0 |
| 2. | Carbopol 934P | 100.0 | 10.0 |
| 3. | Sodium bicarbonate | 30.0 | 3.0 |
| Stage B | | | |
| 1. | Polyvinylpyrrolidone | 12.0 | 1.2 |
| | (K-30) | | |
| 2. | Isopropyl alcohol | Quantity sufficient | q.s |
| Stage C | | | |
| 1. | Carbopol 934P | 50.0 | 5.0 |
| 2. | HPMC K100M | 150.0 | 15.0 |
| 3. | Microcrystalline Cellulose | 113.0 | 11.3 |
| Stage D | | | |
| 1. | PVP K-30 | 10.0 | 1.0 |
| 2. | Isopropyl Alcohol | q.s | q.s |
| Stage E | | | |
| 1. | Talc | 20.0 | 2.0 |
| 2. | Magnesium stearate | 10.0 | 1.0 |
| 3. | Colloidal silicon dioxide | 5.0 | 0.50 |
| | Total | 1000.0 | 100.0 |
| Stage F | | | |
| 1. | Eudragit RL 100 | 37.04 | 3.527 |
| 2. | Isopropyl alcohol | Quantity sufficient | q.s |
| 3. | Acetone | Quantity sufficient | q.s |
| 4. | Triethyl citrate | 3.70 | 0.352 |
| Stage-G | | | |
| 1. | Talcum | 5.56 | 0.529 |
| 2. | Titanium dioxide | 3.70 | 0.352 |
| 3. | Isopropyl alcohol | Quantity sufficient | q.s |

| | | |
|---|---|---|
| Total weight of the coated tablet | 1050 | 100 |

The tablets so obtained were subjected to dissolution testing using USP type I dissolution apparatus. The dissolution medium used was 900 ml of 0.1N HCl for 0-2 hours and 900 ml of simulated intestinal fluid, pH 6.8, for 2-12 hours. The results of the dissolution test are mentioned in Table 4 below.

TABLE 4

| Time (hours) | % drug released (±SD) |
|---|---|
| 1 | 24.63 ± 1.15 |
| 2 | 42.17 ± 3.48 |
| 4 | 61.98 ± 3.5 |
| 8 | 84.97 ± 3.47 |
| 12 | 92.03 ± 3.64 |

### EXAMPLE 3

This Example describes another embodiment of the present invention. The tablets were prepared according to the formula described in Table 5.

Stage A: Metformin Hydrochloride was milled and passed through 100 mesh. Microcrystalline cellulose, xanthan gum, hydroxypropylmethyl cellulose K4M and K100M, were passed through 60 mesh sieve and further uniformly mixed with the drug.

Stage B: The blend was granulated with Methocel E5 dissolved in isopropyl alcohol and water mixture. The wet mass was passed through 20 mesh and the wet granules were dried in Fluidized Bed dryer at 45° C. The dried granules were passed through 30 mesh.

Stage C: All the ingredients of Stage C were passed through 60 mesh and mixed with the dried, sifted granules of metformin hydrochloride. The lubricated granules were compressed at the required tablet weight.

TABLE 5

| | | | % w/w |
|---|---|---|---|
| Stage | | Quantity per dosage | of the |
| Number | Ingredients (mg) | form (mg) | tablet |
| Stage A | | | |
| 1. | Metformin HCL (100#) | 1000.0 | 66.66 |
| 2. | Microcrystalline cellulose | 62.5 | 4.166 |
| 3. | Xanthan gum Type FF | 75.0 | 5.00 |
| 4. | HPMC K4M | 150.0 | 10 |
| 5. | HPMC K100M | 100.0 | 6.666 |
| Stage B | | | |
| 1. | Hydroxypropylmethyl cellulose | 60.0 | 4.00 |
| | (Methocel E5) | | |
| 2. | Isopropyl alcohol | Quantity sufficient | q.s. |
| 3. | Purified Water | Quantity sufficient | q.s. |
| Stage C | | | |
| 1. | Talc | 30.0 | 2.00 |
| 2. | Magnesium stearate | 15.0 | 1.00 |
| 3. | Colloidal Silicon dioxide | 7.50 | 0.50 |

| | | |
|---|---|---|
| Total weight of tablet | 1500.0 | 100 |

The tablets so obtained were subjected to dissolution testing using USP type I dissolution apparatus. The dissolution medium used was 900 ml of 0.1N HCl for 0-2 hours and 900 ml of phosphate buffer pH, for 2-12 hours. The results of the dissolution test are mentioned in Table 6 below.

TABLE 6

| Time (hours) | % drug released (±SD) |
|---|---|
| 1 | 27.55 ± 0.36 |
| 2 | 37.10 ± 0.52 |
| 4 | 57.24 ± 0.76 |
| 8 | 79.50 ± 3.26 |
| 12 | 89.09 ± 1.76 |

### EXAMPLE 4

This Example describes another embodiment of the present invention. The tablets were prepared according to the formula described in Table 7.

Stage A: Metformin Hydrochloride was milled and passed through 100 mesh. Microcrystalline cellulose, xanthan gum, hydroxypropylmethyl cellulose K4M and K100M, were passed through 60 mesh sieve and further uniformly mixed with the drug.

Stage B: The blend was granulated with Methocel E5 dissolved in isopropyl alcohol and water mixture. The wet mass was passed through 20 mesh and the wet granules were dried in Fluidized Bed dryer at 45° C. The dried granules were passed through 30 mesh.

Stage C: All the ingredients of Stage C were passed through 60 mesh and mixed with the dried, sifted granules of metformin hydrochloride. The lubricated granules were compressed at the required tablet weight.

TABLE 7

| | | | % w/w |
|---|---|---|---|
| Stage | | Quantity per dosage | of the |
| Number | Ingredients (mg) | form (mg) | tablet |
| Stage A | | | |
| 1. | Metformin HCL (100#) | 850.0 | 66.66 |
| 2. | Microcrystalline cellulose | 53.125 | 4.166 |
| 3. | Xanthan gum Type FF | 63.750 | 5.00 |
| 4. | HPMC K4M | 127.50 | 10 |
| 5. | HPMC K100M | 85.0 | 6.666 |
| Stage B | | | |
| 1. | Hydroxypropylmethyl cellulose | 51.0 | 4.00 |
| | (Methocel E5) | | |
| 2. | Isopropyl alcohol | Quantity sufficient | q.s |
| 3. | Purified Water | Quantity sufficient | q.s. |
| Stage C | | | |
| 1. | Talc | 25.50 | 2.00 |
| 2. | Magnesium stearate | 12.75 | 1.00 |
| 3. | Colloidal Silicon dioxide | 6.375 | 0.50 |

| | | |
|---|---|---|
| Total weight of tablet | 1275.0 | 100 |

The tablets so obtained were subjected to dissolution testing using USP type I dissolution apparatus. The dissolution medium used was 900 ml of 0.1N HCl for 0-2 hours and 900 ml of phosphate buffer pH 6.8, for 2-12 hours. The results of the dissolution test are mentioned in Table 8 below.

TABLE 8

| Time (hours) | % drug released (±SD) |
|---|---|
| 1 | 29.43 ± 1.00 |
| 2 | 39.06 ± 0.33 |
| 4 | 60.09 ± 0.20 |
| 8 | 81.03 ± 0.53 |
| 12 | 87.04 ± 0.66 |

### EXAMPLE 5

One embodiment of the present invention is described in the following example. The tablets were prepared according to the formula described below:

TABLE 9

| Stage | | Quantity per dosage | % w/w of |
|---|---|---|---|
| Number | Ingredients (mg) | form (mg) | the tablet |
| Stage A | | | |
| 1. | Metformin HCL (100#) | 500.0 | 47.619 |
| 2. | Carbopol 934p | 100.0 | 9.523 |
| 3. | Sodium chloride | 40.0 | 3.809 |
| 4. | HPMC K100M | 180.0 | 17.143 |
| 5. | Microcrystalline cellulose | 50.0 | 4.7619 |
| 6. | Starch | 75.0 | 7.1428 |
| Stage B | | | |
| 1. | Polyvinylpyrrolidone(K-30) | 20.0 | 1.904 |
| 2. | Isopropyl alcohol | Quantity sufficient | q.s |
| 3. | Purified Water | Quantity sufficient | q.s |
| Stage C | | | |
| 1. | Talc | 20.0 | 1.904 |
| 2. | Magnesium stearate | 10.0 | 0.952 |
| 3. | Colloidal Silicon dioxide | 10.0 | 0.952 |
| | Total | 1000.0 | 95.23 |
| Stage D | | | |
| 1. | Eudragit RL 100 | 37.04 | 3.527 |
| 2. | Isopropyl alcohol | Quantity sufficient | q.s |
| 3. | Acetone | Quantity sufficient | q.s |
| 4. | Triethyl citrate | 3.70 | 0.352 |
| Stage-E | | | |
| 1. | Talcum | 5.56 | 0.529 |
| 2. | Titanium dioxide | 3.70 | 0.352 |
| 3. | Isopropyl alcohol | Quantity sufficient | q.s |

| | | |
|---|---|---|
| Total weight of the coated tablet | 1050 | 100 |

Stage A: Metformin hydrochloride and sodium chloride were milled and passed through 100 mesh sieve. Carbopol 934P, hydroxypropylnethyl cellulose, microcrystalline cellulose and starch were passed through 60 mesh sieve and further uniformly mixed with the blend of metformin and sodium chloride.

Stage B: The powder mixture was granulated with polyvinyl pyzrolidone K-30 dissolved in isopropyl alcohol and water mixture. The granules were dried at 45° C. The dried granules were passed through a clit mill.

Stage C: Talc, magnesium stearate and colloidal silicon dioxide were passed through 60 mesh sieve and then mixed with the granules. The lubricated granules were compressed at the required tablet weight.

Stage D & E: The compressed cores were further coated with a polymeric dispersion comprising Eudragit RL100 in isopropyl alcohol and acetone containing triethyl citrate, talcum and titanium dioxide.

The coated tablets were further coated with the coating composition comprising the ingredients described in table 10.

TABLE 10

| Sr. No. | Ingredients | Mg/Tablet | % W/w of the tablet |
|---|---|---|---|
| Stage 1 | | | |
| 1 | HPMC E5 | 17.00 | 15.68 |
| 2 | Isopropyl alcohol | Quantity sufficient | q.s |
| 3 | Methylene chloride | Quantity sufficient | q.s |
| 4 | Glimepiride | 1.00 | 0.0922 |
| 5 | PEG 6000 | 6.80 | 0.6275 |
| 6 | Purified water | Quantity sufficient | q.s |
| Stage 2 | | | |
| 1 | Talcum | 4.40 | 0.406 |
| 2 | Titanium dioxide | 4.40 | 0.406 |
| 3 | Isopropyl alcohol | Quantity sufficient | q.s |

Glimepiride is dissolved in methylene chloride. HPMC is dispersed in isopropyl alcohol. The dispersion is added to the glimepiride solution. PEG 6000 is melted and dissolved in water and added to the glimepiride solution with stirring. Talcum, titanium dioxide and isopropyl alcohol are milled in the colloidal mill and added to the above solution. The solution is mixed well and is used to film coat the coated tablets.

The tablets so obtained were subjected to dissolution testing using USP type I dissolution apparatus. The dissolution medium used was 900 ml of 0.1N HCl for 0-2 hours and 900 ml of simulated intestinal fluid, pH 6.8, for 2-12 hours. The results of the dissolution test are given in Table 11 below.

TABLE 11

| Time (hours) | % metformin released (±SD) |
|---|---|
| 1 | 7.14 ± 0.53 |
| 2 | 28.55 ± 1.05 |
| 4 | 54.44 ± 1.28 |
| 8 | 79.31 ± 1.98 |
| 12 | 88.31 ± 1.32 |

The tablets so obtained were further subjected to dissolution testing using USP type II dissolution apparatus. The dissolution medium used was 900 ml of 0.025 M Tris Buffer pH 9.0 at 50 rpm. The glimepiride release was measured using HPLC. The results of the dissolution test are given in Table 12 below.

TABLE 12

| Time (minutes) | % Glimepiride released (±SD) |
|---|---|
| 10 | 87.14 ± 13.34 |
| 20 | 96.58 ± 20.63 |
| 30 | 99.33 ± 21.27 |
| 45 | 98.64 ± 21.34 |
| 60 | 100.53 ± 21.09 |

### EXAMPLE 6

The tablets containing 500 mg of metformin hydrochloride prepared according to Example 1 were tested in human volunteers in an open label, randomized, comparative and two-way crossover study. A single oral dose containing 500 mg of metformin was administered with 240 ml of water at ambient temperatures. The volunteers fasted overnight before dosing and for 4 hours thereafter. Drinking water was prohibited 2 hours before dosing and 2 hours thereafter. Standard meals were provided at 4 and 8 hours after dosing and at appropriate times thereafter. A wash out period of 7 days was given between the doses. The blood samples were collected before dosing and at 0.5, 1, 2, 3, 3.5, 4, 4.5, 5, 5.5, 6, 6.5, 7, 7.5, 8, 10, 12, 16 and 24 hours and analyzed for metformin.

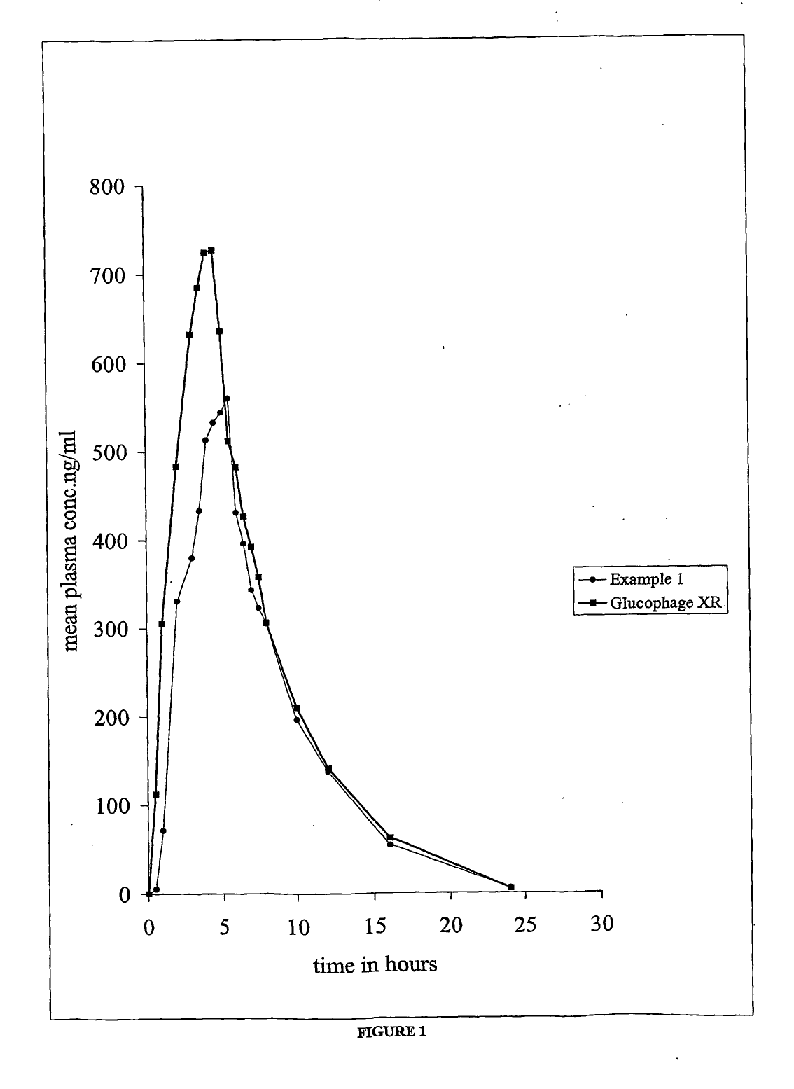

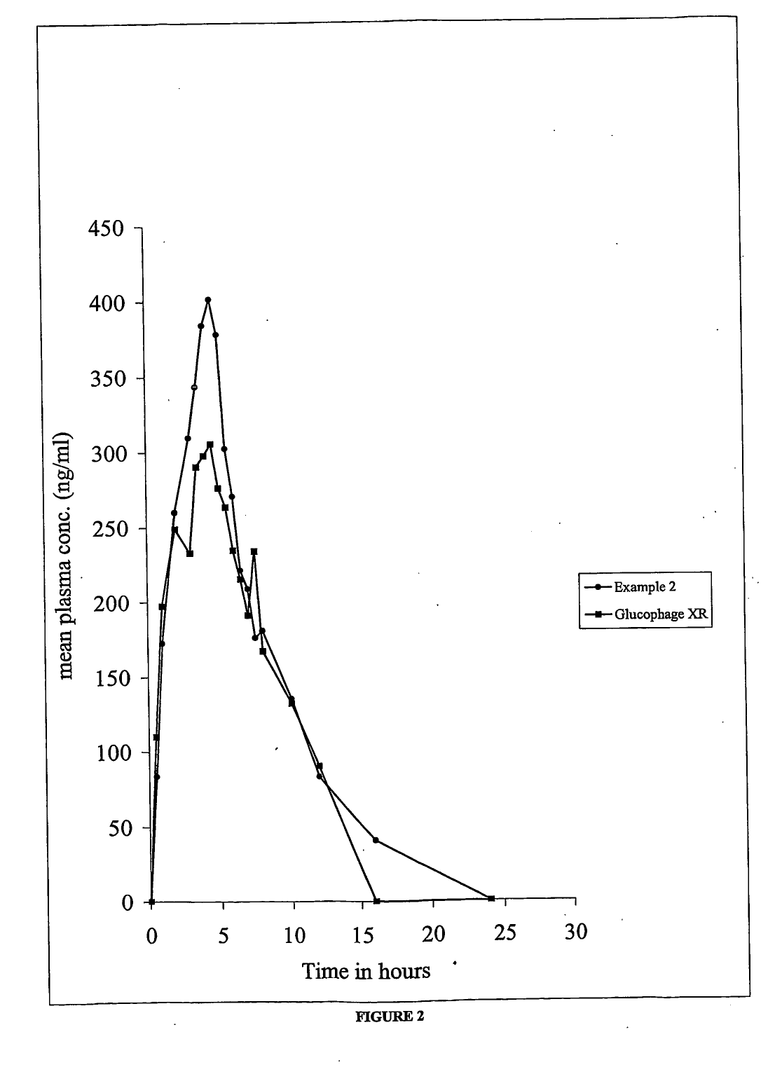

The mean plasma profile given in FIG. 1 demonstrated useful modification of the drug release in vivo. Inter-patient variability in pharmacokinetic parameters was acceptable as illustrated by the % cv given in the Table 13 below.

TABLE 13

| Formulation | Cmax ng/ml | AUC (ng · hr/ml) |
|---|---|---|
| Example 1 | 691.93 (42.36) | 4334.61 (40.96) |
| Glucophage SR 500 mg | 774.35 (29.56) | 5482.19 (31.46) |

### EXAMPLE7

The controlled release antidiabetic composition according to the Example 2 was subjected to pharmacokinetic evaluation as described in Example 6. The pharmacokinetic parameters are tabulated in Table 14. The plasma profile is represented in FIG. 2.

TABLE 14

| Formulation | Cmax ng/ml | AUC (ng · hr/ml) |
|---|---|---|
| Example 2 | 437.02 (49.00) | 3324.90(51.19) |
| Glucophage SR 500 mg | 344.67 (33.96) | 2977.09(88.27) |

1. A dosage form for the treatment of diabetes mellitus and conditions associated with it, comprising a compressible controlled release core composition comprising metformin or its pharmaceutically acceptable salt, two or more swellable polymers wherein at least one polymer is an anionic polymer, one or more pharmaceutically acceptable excipient(s) that improve the compressibility of the core composition, and optionally, a coat comprising one or more water insoluble polymer(s) surrounding the core.

2. A dosage form for the treatment of diabetes mellitus and conditions associated with it as claimed in claim 1 further comprising an immediate release long-acting sulfonyl urea composition.

3. A dosage form as claimed in claim 2 wherein the long-acting sulfonyl urea is selected form a group consisting of carbutamide, chlorpropamide, glyburide (glibenclamide), glimepiride, gliclazide, glibornuride, glyhexamide, phenbutamide, tolazamide, tolbutamide and tolcyclamide.

4. A dosage form as claimed in claim 2 wherein the long-acting sulfonyl urea is glimepiride.

5. A dosage form as claimed in claim 1 wherein the amount of metformin or its pharmaceutical salts is about 500 mg to about 1000 mg per dosage form.

6. A dosage form as claimed in claim 1 wherein the swellable polymer is a high viscosity grade cellulose derivative.

7. A dosage form as claimed in claim 6 wherein the cellulose derivative is hydroxypropyl methyl cellulose (HPMC).

8. A dosage form as claimed in claim 7 wherein a 2% w/w aqueous solution of the hydroxypropyl methyl cellulose has a viscosity in the range from about 80,000 to about 120,000 cps units.

9. A dosage form as claimed in claim 8 wherein the high viscosity grade HPMC is Methocel® K100M.

10. A dosage form as claimed in claim 1 wherein the swellable polymer is a mixture of high viscosity grade HPMC having a viscosity greater than about 10,000 cps for a 2% w/w aqueous solution and a low viscosity grade HPMC having a viscosity equal to or less than about 10,000 cps for a 2% w/w aqueous solution.

11. A dosage form as claimed in claim 10, wherein the high viscosity grade HPMC has viscosity of about 100,000 cps for a 2% w/w aqueous solution and the low viscosity grade HPMC has viscosity of about 4,000 cps for a 2% w/w aqueous solution.

12. A dosage form as claimed in claim 11 wherein the high viscosity grade HPMC is Methocel® K100M and the low viscosity grade HPMC is Methocel® K4M.

13. A dosage form as claimed in claim 1 wherein the anionic polymer is selected from a group of polyacrylic acid or a xanthan gum or mixtures thereof.

14. A dosage form as claimed in claim 1 wherein the concentration of the swellable polymers ranges from about 15 to 40% of the total weight of the said dosage form.

15. A dosage form as claimed in claim 1, wherein the excipient(s) that improve the compressibility of the core composition is selected from a group consisting of microcrystalline cellulose, powdered cellulose, silicified microcrystalline cellulose, dextrins and dextrans, colloidal silicon dioxide, kaolin, titanium dioxide, fused silicon dioxide, alumina, bentonite, magnesium silicate, magnesium trisilicate, anhydrous calcium sulfate, magnesium aluminium silicate and the like.

16. A dosage form as claimed in claim 15 wherein the excipient is microcrystalline cellulose.

17. A dosage form as claimed in claim 1 further comprising excipient(s) that modulate the rate of release of metformin from the core and are selected from a group consisting of osmogents, weak acids and weak bases, surfactants and the like.

18. A dosage form as claimed in claim 17 wherein the osmogent is sodium chloride.

19. A dosage form as claimed in claim 17 wherein the weak base is sodium bicarbonate.

20. A dosage form for the treatment of diabetes mellitus and conditions associated with it comprising an immediate release composition comprising a long-acting sulfonyl urea and a controlled release composition comprising a biguanide.

21. A dosage form as claimed in claim 20 wherein the long-acting sulfonyl urea is glimepiride.

22. A dosage form as claimed in claim 20 wherein the biguanide is metformin or its pharmaceutically acceptable salts.

23. A dosage form as claimed in claim 21 wherein the amount of glimepiride is 1.0 mg.

24. A dosage form as claimed in claim 22 wherein the amount of metformin is 500.0 mg in the form of its base or its pharmaceutically acceptable salt.
